(19) United States
(12) Patent Application Publication
(10) Pub. No.: US 2020/0281971 A1
(43) Pub. Date: Sep. 10, 2020

(54) INHIBITING VIRAL AND BACTERIAL ACTIVITY USING LOW CONCENTRATION HYPOCHLOROUS ACID SOLUTIONS

(71) Applicant: Wonder Spray, LLC, San Diego, CA (US)

(72) Inventor: John Burd, San Diego, CA (US)

(21) Appl. No.: 16/833,209

(22) Filed: Mar. 27, 2020

Related U.S. Application Data
(63) Continuation of application No. 16/293,551, filed on Mar. 5, 2019.

(51) Int. Cl. A61K 33/20; A61P 31/12; A61K 9/00; A61P 31/10; A61K 9/08; A61P 31/04; A61P 33/02

(52) U.S. Cl. CPC A61K 33/20; A61P 31/12; A61K 9/0078; A61P 33/02; A61K 9/08; A61P 31/04; A61P 31/10

(57) ABSTRACT

A method of treating disease states associated with bacteria, a virus, a yeast, a mold, a fungus, a spore, a protozoa or a prion comprising a respiratory, oral, and internal delivery of hypochlorous acid (HOCl) via a nebulizer, an aerosolizer, an spray a spray bottle, an oral rinse, an oral gargle, a vaping device, and a diffuse.

### CROSS-REFERENCE TO RELATED APPLICATIONS

The attached application claims priority to U.S. patent application Ser. No. 16/293,551, filed on Mar. 5, 2019, with the United States Patent and Trademark Office, the contents of which are incorporated herein by reference in its entirety.

### TECHNICAL FIELD

The present disclosure is generally related to inhibiting activity and growth of pathogens, and more specifically, embodiments of the present disclosure relate to the use of hypochlorous acid (HOCl) to inhibit growth of pathogens, including bacteria, virus, yeast, mold, fungus, spore, protozoa or prion.

### BACKGROUND

Antimicrobial medications are commonly used to treat infectious disease. Antimicrobial resistance is a key issue that needs to be taken into account when selecting a therapeutic agent for the treatment of infectious diseases. For example, hospital infections due to multi-resistant bacteria, such as MRSA or Gram-negative multi-resistant bacteria, are serious threats. Although bacterial resistance is a natural phenomenon, the misuse of antimicrobial drugs has accelerated the development of resistance.

Antiseptics agents are known to destroy or inhibit the growth and development of microorganisms in or on living tissue. Unlike antibiotics that act selectively on a specific target, antiseptics have multiple targets and a broader spectrum of activity, which include bacteria, fungi, viruses, yeast, mold, protozoa, spores, archaea, algae, and even prions. Several antiseptic categories exist, including alcohols (ethanol), anilides (triclocarban), biguanides (chlorhexidine), bisphenols (triclosan), chlorine compounds, iodine compounds, silver compounds, peroxygens, and quaternary ammonium compounds. In particular, chlorine-based compounds have been traditionally used for both antiseptic and disinfectant purposes.

### SUMMARY

In accordance with one or more embodiments, various features and functionality can be provided to enable or otherwise facilitate delivery of HOCl.

In some embodiments, the method of treating disease states associated with an increased microbial activity comprises delivering an antiseptic solution of hypochlorous acid to a patient via a respirable delivery method.

In some embodiments, the method of treating disease states associated with an increased microbial activity comprises delivering an antiseptic solution of hypochlorous acid having a pH range of approximately 6.1 to 6.3.

In some embodiments, the method of treating disease states associated with an increased microbial activity comprises placing the aqueous solution of the hypochlorous acid into a reservoir of a nebulizer. In some embodiments, the method of treating disease states associated with an increased microbial activity comprises aerosolizing the aqueous solution of hypochlorous acid into particles. In some embodiments, the hypochlorous acid is aerosolized into particles sized approximately from 0.1 μm to 99 μm. In some embodiments, the method of treating disease states associated with an increased microbial activity comprises administering aerosolized particles of the aqueous solution of hypochlorous acid through a mouth piece affixed to the nebulizer into an upper respiratory track of the patient.

In some embodiments, the method of treating disease states associated with an increased microbial activity comprises placing the aqueous solution of the hypochlorous acid within a reservoir of a spray bottle. In some embodiments, the method of treating disease states associated with an increased microbial activity comprises transforming the aqueous solution of the hypochlorous acid into a mist. In some embodiments, the method of treating disease states associated with an increased microbial activity comprises spraying the mist of the aqueous solution of hypochlorous acid into one or more nasal passages and sinuses cavities of the patient.

In some embodiments, the method of treating disease states associated with an increased microbial activity caused by at least one of Acinetobacter baumannii, Bacillus subtilis, Enterobacter cloacae, Enterococcus faecalis, Escherichia coli, Escherichia coli, Escherichia coli, Enterobacter, Klebsiella pneumoniae, Listeria monocytogenes, MRSA (Staph. aureus), Polymicrobial biofilm, Proteus vulgaris, Pseudomonas aeruginosa, Salmonella choleraesuis, Shigella flexneri, Staph epidermidis, and Yersinia enterocolitica.

In some embodiments, the method of treating disease states associated with an increased microbial activity caused by at least one of a bacteria, a virus, a yeast, a mold, a fungus, a spore, a protozoa or a prion.

### BRIEF DESCRIPTION OF THE DRAWINGS

FIG. 1 illustrates a perspective view of an antiseptic agent delivery system for administering HOCl comprising a nebulizer, according to an implementation of the disclosure.

FIG. 2 illustrates a process of administering HOCl using a nebulizer, according to an implementation of the disclosure.

### DETAILED DESCRIPTION

Hypochlorous acid (HOCl) is a weak acid that forms when chlorine dissolves in water, and itself partially dissociates, forming hypochlorite, OCl—. Similar to other chlorine-releasing agents (e.g., sodium hypochlorite, chlorine dioxide, and the N-chloro compounds such as sodium dichloroisocyanurate), aqueous chlorine solution is well known for its antimicrobial, anti-inflammatory, and immunomodulatory properties.

Applications of aqueous solutions containing approximately 30-2500 ppm (0.003% to 0.25%) HOCl are used in a variety of areas including (but not limited to) wound care, as antimicrobial agents, as anti-allergen agents, dental care and there are also significant applications in water treatments, food sanitization, and hard surface disinfection, and cosmetics.

HOCl is a potent antimicrobial capable of eradicating bacteria including antibiotic-resistant strains, viruses, fungi, and spores. In particular, HOCl is the active component responsible for pathogen disruption and inactivation by chlorine-releasing agents (CRAB). It is understood that the OCl-ion has little effect compared to undissolved HOCl, as the hypochlorite (OCl-), has only a minute effect compared to undissolved HOCl. Accordingly, the microbicidal effect of HOCl is the greatest when the percentage of undissolved HOCl is highest. In an aqueous solution of HOCl, ranging from approximately pH 4 to pH 7, chlorine exists predominantly as HOCl, whereas above pH 9, ClO— predominates.

Because HOCl is a highly active oxidizing agent, its mode of operation comprises destroying and/or deactivating cellular activity of proteins. For example, HOCl targets bacteria by chemically linking chlorine atoms to nucleotide bases that disrupt the function of bacterial DNA, impede metabolic pathways in which cells use enzymes to oxidize nutrients, and release energy, and other membrane-associated activities. Additionally, HOCl has also been found to disrupt oxidative phosphorylation and other membrane-associated activity. Similarly, HOCl has been found to inhibit bacterial growth. For example, at 50 mM (2.6 ppm), HOCl completely inhibited the growth of E. coli within 5 minutes, including inhibiting the DNA synthesis by ninety-six percent. Unlike conventional antibiotics, the antimicrobial activity of HOCl is directly toxic to microbial cells, including many Gram-positive and Gram-negative bacteria and their biofilms. HOCl has demonstrated disinfection efficacy against eradication of bacteria, including Acinetobacter baumannii, Bacillus subtilis, Enterobacter cloacae, Enterococcus faecalis, Escherichia coli, Escherichia coli, Escherichia coli, Enterobacter, Klebsiella pneumoniae, Listeria monocytogenes, MRSA (Staph. aureus), Polymicrobial biofilm, Proteus vulgaris, Pseudomonas aeruginosa, Salmonella choleraesuis, Shigella flexneri, Staph epidermidis, and Yersinia enterocolitica.

Additionally, HOCl possesses viricidal activity properties. For example, it has been demonstrated that HOCl inactivated naked f2 RNA at the same rate as RNA in intact phage, whereas f2 capsid proteins could still adsorb to the host. HOCl has demonstrated disinfection efficacy against eradication of viruses including norovirus, filoviruses such as Ebola, and human coronaviruses like MERS-CoV and SARS, as well as fungi such as Candida and Aspergillus. Further, as a sporicide, HOCl causes the spore coat to detach from the cortex, where further degradation occurs.

Both topical and internal applications of HOCl are safe because it is the exact same substance white blood cells in the human body produce to fight infection. Indeed, extensive studies have demonstrated exceptional safety of HOCl. The Food and Drug Administration (FDA) has approved preparations of HOCl to be used, e.g., topically for eye infections, tooth infections, nasal decontamination, and the care of surgical incisions. In particular, inhaling the aerosolized form of HOCl has also been shown to causes no adverse effects.

The advent of antibiotics and other area disinfectants led to a reduction in environmental use of HOCl. However, widespread use of antibiotic agents led to antimicrobial resistance. Accordingly, an urgent need to optimize currently available anti-infectious therapies to overcome drug resistance exists.

Embodiments of the technology disclosed herein are directed to antiseptic agent delivery systems in which the antiseptic agent is administered via a pulmonary route as a treatment of infectious diseases caused by microbial (including spores), viral, fungal, allergy-causing agents. Because the inhalation process gives a more direct access to the target organ/cavity than more traditional routes (e.g., topical, oral, intravenous, etc.), the pulmonary administration of HOCl used to inhibit bacterial growth provides a therapeutic approach that may help avoid reduce antimicrobial resistance while alleviating the disease symptoms. For example, upper respiratory tract infections caused by one or more bacterial or viral pathogens such as bronchitis, epiglottitis, laryngitis, sinusitis, rhinosinusitis, chronic rhinosinusitis and so on, lung infections, such as pneumonia, may be treated by a pulmonary administration of an antiseptic agent, such as HOCl.

In some embodiments, a solution of HOCl may be delivered via the pulmonary route via a number pulmonary delivery devices. For example, the HOCl may be delivered via a nebulizer, an aerosolizer, atomizer, and/or any other such pulmonary delivery device. For example, a solution of HOCl of low concentration levels and relatively low acidotic pH may be used as a nebulized topical laryngeal, tracheal, and alveolar disinfectant. In some embodiments, the aqueous solution of HOCl may include a concentration of approximately 0.01 percent of HOCl dissolved in water.

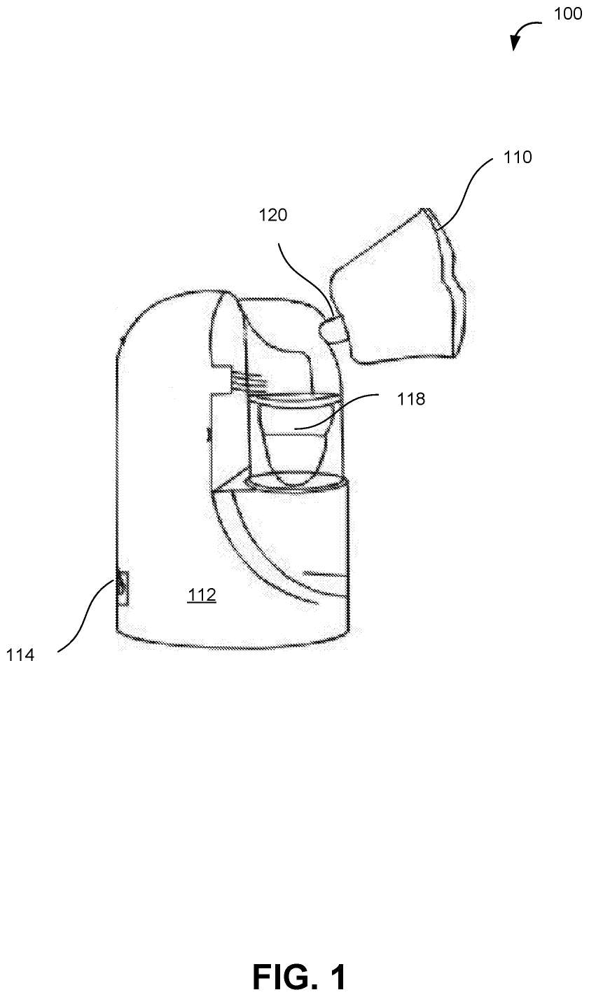

FIG. 1 depicts an antiseptic agent delivery system for delivering HOCl via the pulmonary route. The antiseptic agent delivery system 100 or components/features thereof may be implemented in combination with, or as an alternative to, other systems/features/components described herein, such as those described with reference to other embodiments and figures. The antiseptic agent delivery system 100 may additionally be utilized in any of the methods for using such systems/components/features described herein. The antiseptic agent delivery system 100 may also be used in various applications and/or permutations, which may or may not be noted in the illustrative embodiments described herein. For instance, antiseptic agent delivery system 100 may include more or less features/components than those shown in FIG. 1, in some embodiments. Moreover, the antiseptic agent delivery system 100 is not limited to the size, shape, number of components, etc. specifically shown in FIG. 1.

As shown in FIG. 1, the antiseptic agent delivery system 100 comprises a housing 112 which houses one or more components configured to aerosolize the aqueous antiseptic solution so that it can be administered it in the form of aerosolized particles by being inhaled into lungs. For example, the one or more components hosed in housing 112 may include an ultrasonic generator or oscillator, a compressor, or similar components and associated circuitry (not shown) for causing aerosolization.

Further, the antiseptic agent delivery system 100 comprises a liquid supply reservoir 118 and a mouth piece 110. In some embodiments, the aqueous antiseptic solution may be placed within the liquid supply reservoir 118. For example, the aqueous antiseptic solution may include liquid HOCl liquid solution ranging from 0.5 ml to 20 ml placed in the reservoir 118.

In some embodiments, the one or components housed within housing 112 may cause the aqueous antiseptic solution to be aerosolize the aqueous antiseptic solution. For example, the housing 112 may include an inlet (not shown) through which air is supplied under pressure from a compressor (not shown). In some embodiments, agent delivery system 100 may be configured to use a driving gas flow (typically 0.5 mL/min) to generate aerosol. For example, the nebulizers may deliver an approximately equal volume of aerosol during the inhalation phase (i.e., when patient is breathing). In other embodiments, an oscillator (not shown) may transmit ultrasonic waves through the aqueous antiseptic solution.

In some embodiments, the pressurized air may be directed via an air channel (not shown) into the liquid supply reservoir 118 causing a rapid formation and collapse of bubbles, which then stream toward the surface of the solution and encounter the interface between the solution and air, resulting in a production of a fine mist or aerosol adjacent the solution surface.

In some embodiments, the antiseptic agent delivery system 100 may be configured to carry the aerosol upwardly through a conduit 120 connected to the mouth piece 110. The patient may aspirate the aerosolized aqueous antiseptic solution through the mouth piece 110. In some embodiments, the diameter of aerosol particles or droplets may be approximately 1 to 5 microns to ensure the particles or droplets are not likely to be impacted in the airway before they reach the lungs and are not carried out of the lungs again on exhalation without being deposited within the respiratory system structures (e.g., lungs).

In some embodiments, the conduit 120 may be configured to be slightly larger in diameter than an exit port (not shown) within the mouthpiece 110. By virtue of the conduit 120 being slightly larger in diameter than the air exit port of the mouth piece 100 a small space between the outer surface of the air exit port and the inner surface of the conduit 120 is provided. For example, the space may be approximately 0.00254-0.254 mm. In some embodiments, the space allows fluid from the liquid supply reservoir 118 to proceed upward between the air exit port and the conduit 120. In some embodiments, The diameter of the conduit 120 may be adjusted to change the particle size of the mist.

In some embodiments, the housing 112 may include one or more pressure sensors (not shown) configured to detect the pressure within the liquid supply reservoir 118. In some embodiments, the one or more pressure sensors may be connected to the inside of the mouth piece 100. In some embodiments, the one or more pressure sensors may detect that a patient has inhaled causing the antiseptic agent delivery system 100 to divert pressurized air to an air outlet (not shown).

In some embodiments, the antiseptic agent delivery system 100 may be configured to analyze the pressure changes within the system 100 during a certain number of initial breaths (e.g., first three breaths) to determine an average shape of the breathing pattern. A timed pulse of atomization is commenced upon start of subsequent inspirations such that atomization occurs for the first 50 percent of the inspiration. In some embodiments, the antiseptic agent delivery system 100 may be configured to have a timed pulse of atomization to occur during a period other than 50% of the duration of inspiration. In some embodiments, the antiseptic agent delivery system 100 may be configured to have a predetermined pulse length. For example, the pulse length may be set for each patient by a clinician.

In some embodiments, the housing 112 may include one or more panels (not shown) to operate the one or more components configured to aerosolize the aqueous antiseptic solution. In some embodiments, the housing 112 may comprise a connector 114 to which a power cable (not shown) may be connected.

In some embodiments, the nebulized HOCl may be delivered using compressor-based jet-nebulizer system. For example, a jet nebulizer may be configured with a mechanism to allow the production of aerosol HOCl only when the inhalation airflow exceeds a certain flow rate. By virtue of including the mechanism, the jet nebulizer provides control over the portion of the breath into which the aerosol HOCl is delivered.

In some embodiments, the nebulized HOCl may be delivered using a mesh-based nebulizer system. In some embodiments, the mesh-based nebulizer may be used to deliver aerosol HOCl based on a breathing-pattern of a patient. For example, the mesh-based nebulizer may include one or more sensors configured to monitor inspiratory flow rate and length of the inhalation. In some embodiments, aerosol HOCl may be pulsed during the first fifty to eighty percent of the inhalation, based on determined specific characteristics of the breathing pattern. The duration of each pulse of aerosol HOCl may be determined by the patient's breathing pattern and varied for each subsequent breath, depending on the preceding breaths.

In some embodiments, the nebulized HOCl may be delivered using an ultrasonic wave nebulizer. For example, the ultrasound wave nebulizer may comprise an electronic oscillator and a one or more piezoelectric elements to create an aerosol. The electronic oscillator may be configured to generate a high frequency ultrasonic wave, which causes the mechanical vibration of the one or more piezoelectric elements, the one or more piezoelectric elements may be in contact with a compartment used to store an aqueous HOCl solution. The one or more piezoelectric elements may vibrate at a high frequency and deliver a vapor mist comprising an aerosolized HOCl.

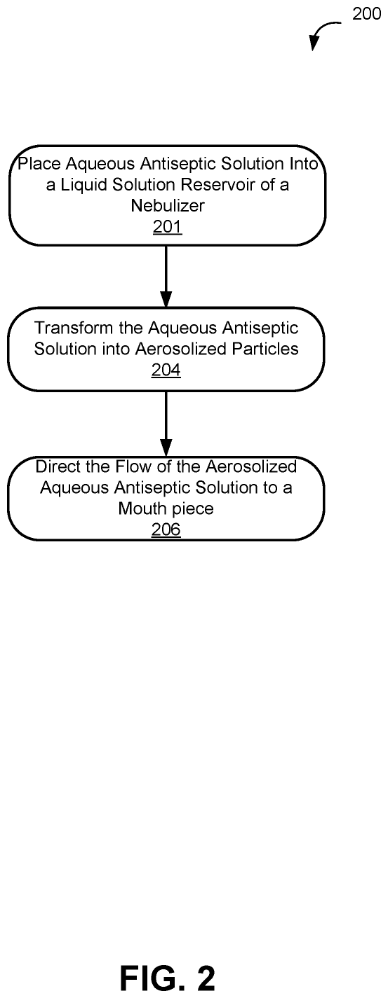

In some embodiments, illness suspected to be caused by microbial (including spores), viral, fungal, allergy-causing agents may be treated by administering HOCl through use of aerosolized or atomized pulmonary administration. Embodiments using this method involve the use of an aerosolizer or an atomizer to aerosolize a liquid HOCl solution for respirable delivery. For example, and as illustrated in FIG. 2, the delivery of the aerosolized HOCl may include one or more of the following operations. In an operation 202, an aqueous antiseptic solution may be placed in a reservoir of an aerosolizing, atomizing, or similar device. For example, the aqueous antiseptic solution comprising HOCl ranging in volume from approximately 0.5 ml to 10 ml may be placed into the reservoir. In an operation 204, the aqueous antiseptic solution may be aerosolized into particle sizes. For example, the aqueous antiseptic solution comprising HOCl may be aerosolized into particles ranging from approximately 0.1 μm to 99 μm or larger, in size. In an operation 206, the aqueous antiseptic solution may be directed into a mouth piece affixed to the device to be aspired by a patient into their upper respiratory track. For example, the aerosolized aqueous antiseptic solution comprising HOCl may be aspired for a prescribed duration (e.g., a period ranging from approximately 0.5 min to 30 min. or longer).

In some embodiments, illness suspected to be caused by microbial (including spores), viral, fungal, allergy-causing agents may be treated by administering HOCl through use of aerosolized or atomized administration via patient's nasal sinus passages. For example, the delivery of the aerosolized HOCl may include one or more of the following: placing liquid solution ranging from 0.5 ml to 10 ml placed in the reservoir of an aerosolizing, atomizing, or similar device, aerosolizing or atomizing the liquid into particle sizes ranging from approximately 0.1 μm to 99 μm or larger, and inserting a nose piece affixed to the device into the nostril(s) for a prescribed duration (e.g., a period ranging from approximately 0.5 min to 10 min, or longer).

In some embodiments, illness suspected to be caused by microbial (including spores), viral, fungal, allergy-causing agents may be treated by administering HOCl through use of a nasal spray. For example, a spray device comprising a pump and a spray nozzle, may be used to transform the aqueous solution of HOCl into a mist for administering the solution to the nasal passages and sinus cavities. In some embodiments, the delivery of the mist comprising an aqueous HOCl solution may include one or more of the following: placing an aqueous solution of HOCl ranging from 0.1 ml to 10 ml into a nasal spray bottle adequately suited for nasal use, and administering the mist comprising aqueous solution HOCl into the nasal passages of each nostril via the spray bottle by compressing the spray bottle. For example, approximately one to ten sprays per nostril may be administered into each nostril by compressing the spray bottle. In some embodiments, the aqueous solution of HOCl may be diluted with one or more diluents. For example, 0.5 ml to 20 ml of saline may be added to the aqueous solution of HOCl.

In some embodiments, illness suspected to be caused microbial (including spores), viral, fungal, allergy-causing agents may be treated by administering HOCl through use of nasal rinsing or irrigation. Generally, nasal rinses and irrigation systems are used to flush out excess mucus and debris from the nasal passages and sinus cavities, but they can also be used to administer medicated solutions to the nasal passages and sinus cavities. In some embodiments, the delivery of the aqueous solution of HOCl may include one or more of the following: adding an aqueous solution of HOCl (ranging approximately from approximately 0.1 ml to 10 ml) to a commercially available or prepared nasal rinsing or irrigation solution, and administering the aqueous solution of HOCl to the nasal passages and sinus cavities by rinsing, flushing, irrigating, or otherwise exposing nasal passages and cavities to the combined aqueous solution of HOCl and rinsing or irrigating solution.

In some embodiments, illness suspected to be caused microbial (including spores), viral, fungal, allergy-causing agents may be treated by administering HOCl through use of an oral rinse solution. Generally, oral rinses use a liquid solution that is used to swish around the oral cavity, including teeth, gums and tongue to help prevent or treat various oral health conditions and diseases (e.g., gum disease, halitosis, gingivitis, tartar, and so on). In some embodiments, the aqueous solution of HOCl may be diluted with one or more diluents. For example, approximately 0.5 ml to 20 ml of saline may be added to the aqueous solution of HOCl. In some embodiments, a particular amount of aqueous solution of HOCl (e.g., approximately 5 mL) may be placed inside a patient's oral cavity without swallowing. While keeping the lips closed, the patient may make a swishing motion to move the HOCl so that so that the HOCl solution reaches the front and sides of your mouth equally for a prescribed duration (e.g., a period ranging from approximately 0.5 min to 10 min, or longer), repeating it as necessary. Upon completing the oral rinse, the HOCl solution may be spitted out.

In some embodiments, illness suspected to be caused microbial (including spores), viral, fungal, allergy-causing agents may be treated by administering HOCl through use of a gargling solution. For example, a particular amount of aqueous solution of HOCl (e.g., approximately 5 mL) may be placed inside a patient's oral cavity without swallowing. While keeping the lips and teeth slightly apart and tilting the head slightly backwards, the patient may move the liquid within the throat cavity by exhaling through it for a prescribed duration (e.g., a period ranging from approximately 0.5 min to 10 min, or longer), repeating it as necessary. Upon completing the gargling, the HOCl solution may be spitted out. In some embodiments a spray device comprising a spray nozzle, may be used to transform the aqueous solution of HOCl into a mist for administering the solution to the oral cavity and/or throat structures (e.g., oropharynx, larynx, etc.). In some embodiments, the aqueous solution of HOCl may be diluted with one or more diluents. For example, approximately 0.5 ml to 20 ml of saline may be added to the aqueous solution of HOCl.

In some embodiments, illness suspected to be caused by adverse reactions to one or more medications and/or lifestyle choices may be may be treated by administering HOCl through use of an or oral rinse and/or gargling solution, as described above. For example, oral inflammation and/or ulceration (e.g., mucositis) which may arise as an adverse effect to a particular medication (e.g., chemotherapy and radiotherapy treatment for cancer) or due to dehydration, poor mouth care, oxygen therapy, excessive use of alcohol and/or tobacco, and lack of protein in the diet may be treated by placing a particular amount of aqueous solution of HOCl (e.g., approximately 5 mL) may be placed inside a patient's oral cavity without swallowing and used as an oral rinse and/or a gargling. In some embodiments, the aqueous solution of HOCl may be diluted with one or more diluents. For example, approximately 0.5 ml to 20 ml of saline may be added to the aqueous solution of HOCl.

In some embodiments, the delivery of the mist comprising an aqueous HOCl solution may include one or more of the following: placing an aqueous solution of HOCl ranging from 0.1 ml to 10 ml into a spray bottle adequately suited for oral use, and administering the mist comprising aqueous solution HOCl into the oral cavity and surrounding throat structures via the spray bottle by compressing the spray bottle. For example, approximately one to ten sprays may be administered into the oral cavity by compressing the spray bottle. In some embodiments, the aqueous solution of HOCl may be diluted with one or more diluents. For example, approximately 0.5 ml to 20 ml of saline may be added to the aqueous solution of HOCl.

In some embodiments, one or more effects of relaxing one or more respiratory structures (e.g., uvula, soft palate, etc.) resulting in a sound (e.g., snoring) due to their vibrations during sleep may be treated by administering HOCl. For example, the HOCl may be administered through the use of a HOCl containing oral rinse solution, a gargling solution, as described above.

In some embodiments, an irritation, inflammation, and/or obstruction of the breathing passages resulting in in a cough reflex and often associated with acute and/or chronic respiratory tract infection may be treated by administering HOCl. For example, the HOCl may be administered through the use of a HOCl containing oral rinse solution, a gargling solution, as described above. In some embodiments, the HOCl solution may be administered by a pulmonary delivery method, as described above.

In some embodiments, an irritation and/or an inflammation of the voice box resulting in loss of voice and/or diminished capacity to produce sound (e.g., laryngitis) may be treated by administering HOCl. For example, the HOCl may be administered through the use of a HOCl containing oral rinse solution, a gargling solution, as described above. In some embodiments, the HOCl solution may be administered by a pulmonary delivery method, as described above.

In some embodiments, an irritation and/or an inflammation of one or more structures within the nasal cavity and/or throat due to an allergic reaction to one or more allergens, such as pet dander, dust, mites, pollen and mold, may be treated by administering HOCl. For example, the HOCl solution may be used to decrease the activity of allergy-causing agents through the use of a HOCl containing nasal spray solution, a nasal rinse or irrigation solution, an oral rinse solution, and/or a gargling solution, as described above. In some embodiments, the HOCl solution may be administered by a pulmonary delivery method, as described above. Alternatively, the HOCl solution may be used to decrease the histamine response which may be elevated during an allergic response to one or more allergens, as previously alluded. For example, the HOCl may be administered through the use of a HOCl containing oral rinse solution, a gargling solution, as described above, or a pulmonary delivery method.

In some embodiments, the pH level of the HOCl solution administered through the methods disclosed herein may be pH-neutral because stabilized and/or pH-neutral HOCl is superior in terms of antimicrobial activity to non-stabilized HOCl and acidified bleach, including against hypochlorite-resistant strains. In some embodiments, the acidotic pH level of the HOCl may be within the range resulting in the highest amount of undissolved HOCl. For example, the acidotic pH level may range from approximately pH 6.1 to approximately pH 6.3.

In some embodiments, illness suspected to be caused by microbial (including spores), viral, fungal, allergy-causing agents may be treated by administering HOCl through use of a vaping device. The vaping device may comprise a cartridge configured to store HOCl solution, and a heating element/atomizer, a microprocessor, a battery, and/or other such similar components. In some embodiments, the delivery of the vapor solution of HOCl may include one or more of the following: adding an aqueous solution of HOCl (ranging approximately from approximately 0.1 ml to 10 ml) to a cartridge of a vaping device, atomizing the liquid into particle sizes ranging from approximately 0.1 μm to 99 μm or larger by the heating element, and breathing the atomized particles through a mouth piece affixed to the device into the upper respiratory track to administer the atomized HOCl for a prescribed duration (e.g., a period ranging from approximately 0.5 min to 30 min. or longer). In some embodiments, HOCl solution may be heated to a certain temperature (e.g., at or about 100-250° C.) to create an aerosolized vapor.

In some embodiments, illness suspected to be caused by microbial (including spores), viral, fungal, allergy-causing agents may be treated by administering HOCl solution as an agent in an aerial diffusion. For example, disinfectant properties of HOCl may be delivered via a diffusion device. In some embodiments a diffusion device may be configured to volatilize HOCl into the air. The volatilized HOCl may then be inhaled by one or more patients to treat one or more respiratory illness suspected to be caused by microbial (including spores), viral, fungal, allergy-causing agents.

In some embodiments, ophthalmic illnesses suspected to be caused by microbial (including spores), viral, fungal, allergy-causing agents may be treated by administering HOCl solution. For example, disinfectant properties of HOCl may be delivered via a dropper or a similar device adapted for delivering solutions into a patient's eye.

In some embodiments, cochlear illnesses suspected to be caused by microbial (including spores), viral, fungal, allergy-causing agents may be treated by administering HOCl solution. For example, disinfectant properties of HOCl may be delivered via be delivered via a dropper or a similar device adapted for delivering solutions into a patient's ears.

In some embodiments a diffusion device may be configured to volatilize HOCl into the air. The volatilized HOCl may then be inhaled by one or more patients to treat one or more respiratory illness suspected to be caused by microbial (including spores), viral, fungal, allergy-causing agents.

In some embodiments, HOCl solution may be administered intravenously and used as a prophylactic solution to defend against a potential microbial (including spores), viral, fungal, allergy-causing agents. For example, HOCl solution, administered intravenously, may be used to counter one or more types of influenza virus (e.g., H1N1) strains.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method of destroying coronavirus in an affected area, the method comprising:
delivering an antiseptic solution to the affected area, wherein the antiseptic solution comprises an aqueous solution of hypochlorous acid.

2. The method of claim 1, wherein the hypochlorous acid has a pH range of approximately 6.1 to 6.3.

3. The method of claim 1, wherein the delivery of antiseptic solution comprises:
placing the aqueous solution of the hypochlorous acid into a reservoir of a nebulizer;
aerosolizing the aqueous solution of hypochlorous acid into particles; and
distributing the aerosolized particles of the aqueous solution of hypochlorous acid into the affected area.

4. The method of claim 3, wherein the particles are approximately from 0.1 μm to 99 μm in size.

5. The method of claim 1, wherein the delivery of antiseptic solution comprises:
placing the aqueous solution of the hypochlorous acid within a reservoir of a spray bottle;
transforming the aqueous solution of the hypochlorous acid into a mist; and
spraying the mist of the aqueous solution of hypochlorous acid into the affected area.

6. The method of claim 1, wherein the delivery of antiseptic solution comprises:
placing the aqueous solution of the hypochlorous acid within a reservoir of an atomizer;
transforming the aqueous solution of the hypochlorous acid into a fine spray; and
distributing the fine spray of the aqueous solution of hypochlorous acid into the affected area.

7. A method of deactivating coronavirus in an affected area, the method comprising:
delivering an antiseptic solution to the affected area, wherein the antiseptic solution comprises an aqueous solution of hypochlorous acid.

8. The method of claim 7, wherein the hypochlorous acid has a pH range of approximately 6.1 to 6.3.

9. The method of claim 7, wherein the delivery of antiseptic solution comprises:
placing the aqueous solution of the hypochlorous acid into a reservoir of a nebulizer;
aerosolizing the aqueous solution of hypochlorous acid into particles; and
distributing the aerosolized particles of the aqueous solution of hypochlorous acid into the affected area.

10. The method of claim 9, wherein the particles are approximately from 0.1 μm to 99 μm in size.

11. The method of claim 7, wherein the delivery of antiseptic solution comprises:
placing the aqueous solution of the hypochlorous acid within a reservoir of a spray bottle;
transforming the aqueous solution of the hypochlorous acid into a mist; and
spraying the mist of the aqueous solution of hypochlorous acid into the affected area.

12. The method of claim 7, wherein the delivery of antiseptic solution comprises:
placing the aqueous solution of the hypochlorous acid within a reservoir of an atomizer;
transforming the aqueous solution of the hypochlorous acid into a fine spray; and
distributing the fine spray of the aqueous solution of hypochlorous acid into the affected area.

13. A method of disinfecting an area affected by coronavirus, the method comprising:
delivering an antiseptic solution to the area affected by the coronavirus, wherein the antiseptic solution comprises an aqueous solution of hypochlorous acid.

14. The method of claim 13, wherein the hypochlorous acid has a pH range of approximately 6.1 to 6.3.

15. The method of claim 13, wherein the delivery of antiseptic solution comprises:
placing the aqueous solution of the hypochlorous acid into a reservoir of a nebulizer;
aerosolizing the aqueous solution of hypochlorous acid into particles; and
distributing the aerosolized particles of the aqueous solution of hypochlorous acid into the area affected by the coronavirus.

16. The method of claim 15, wherein the particles are approximately from 0.1 μm to 99 μm in size.

17. The method of claim 13, wherein the delivery of antiseptic solution comprises:
placing the aqueous solution of the hypochlorous acid within a reservoir of a spray bottle;
transforming the aqueous solution of the hypochlorous acid into a mist; and
spraying the mist of the aqueous solution of hypochlorous acid into the area affected by the coronavirus.

18. The method of claim 13, wherein the delivery of antiseptic solution comprises:
placing the aqueous solution of the hypochlorous acid within a reservoir of an atomizer;
transforming the aqueous solution of the hypochlorous acid into a fine spray; and
distributing the fine spray of the aqueous solution of hypochlorous acid into the area affected by the coronavirus.
